(19) United States
(12) Patent Application Publication
(10) Pub. No.: US 2025/0073180 A1
(43) Pub. Date: Mar. 6, 2025

(54) COMPOSITIONS COMPRISING NON-RACEMIC MIXTURES OF R- AND S-1,3-BUTANEDIOL AND USES THEREOF

(71) Applicant: Nicholas Kadysh, Toronto (CA)

(72) Inventor: Nicholas Kadysh, Toronto (CA)

(21) Appl. No.: 18/285,787

(22) PCT Filed: May 10, 2023
(86) PCT No.: PCT/CA2023/050639

§ 371 Date: Oct. 5, 2023

Related U.S. Application Data
(60) Provisional application No. 63/340,673, filed on May 11, 2022.

(51) Int. Cl. A61K 31/047; A61P 3/02; C07C 31/20

(52) U.S. Cl. CPC A61K 31/047; A61P 3/02; C07C 31/207

(57) ABSTRACT

The present application includes a composition comprising a non-racemic mixture R-1,3-butanediol and S-1,3-butane-diol, wherein the R-1,3-butanediol is present in the composition in a greater amount by enantiomeric equivalents, relative to S-1,3-butanediol, and uses thereof.

### RELATED APPLICATIONS

The present application claims the benefit of priority of co-pending U.S. provisional patent application No. 63/340,673 filed on May 11, 2022, the contents of which are incorporated herein by reference in their entirety.

### FIELD

The present application relates to compositions comprising non-racemic mixtures of 1,3-butanediol and methods of using these compositions, for example, as an ethanol replacement.

### BACKGROUND

A beverage that comprises at least 0.5% by volume of D (or R)-ethyl-3-hydroxybutyrate and/or D (or R)-1,3-butanediol is described in US 2019/0177673. In this patent application, it is reported that D-ethyl 3-hydroxybutyrate and D-1,3-butanediol have each been shown to be safe for human consumption and further that D-ethyl-3-hydroxybutyrate, and D-1,3-butanediol can each be converted into β-hydroxybutyrate and can serve as a substrate for brain metabolism. In addition, D-ethyl-3-hydroxybutyrate, and D-1,3-butanediol are hypoglycemic agents, meaning they can reduce glucose levels in the blood. Moreover, when used, e.g., as an alcohol in a beer, they can help initiate and/or maintain ketosis.

Ketosis is a metabolic state characterized by elevated levels of ketone bodies in the blood or urine. Ketone bodies are water-soluble molecules that contain the ketone groups produced from fatty acids by the liver (ketogenesis). Physiologic ketosis is a normal response to low glucose availability, such as low-carbohydrate diets or fasting, that provides an additional energy source for the brain in the form of ketone bodies. In physiologic ketosis, ketones in the blood are elevated above baseline levels, but the body's acid-base homeostasis is maintained. An elevated level of ketone bodies in the blood has been shown to reduce the frequency of epileptic seizures, as well to improve brain function when a person's body cannot properly use glucose, such as in Alzheimer's patients and those with concussions or other brain damage. Ketone bodies may also improve muscle performance, such as in endurance athletes.

D-3-Hydroxybutyrate is one of the most commonly studied ketone body. Little attention has been paid to L-3-hydroxybutyrate because it is generally found in only small amounts. However, studies have shown that the concentrations of L-3-hydroxybutyrate are the highest in heart tissues of rats and it has been suggested that L-3-hydroxybutyrate is not utilized for energy fuel as other ketone bodies are (Tsai, Y.-C. et al. Life Sciences. 2006, 78:1385-1391). In addition, observed reduced glucose utilization caused by D-3-hydroxybutyrate gradually recovered in a dose-dependent manner with administration of additional L-3-hydroxybutyrate.

### SUMMARY

The present application includes a composition comprising a non-racemic mixture of R-1,3-butanediol and S-1,3-butanediol wherein R-1,3-butanediol is present in the composition in a greater amount by enantiomeric equivalents, relative to S-1,3-butanediol.

The present application further includes a consumable product comprising a composition of the application.

In some embodiments, the consumable product is a beverage. In some embodiments, the beverage is an alcoholic beverage.

In some embodiments, the consumable product is a nutraceutical.

The present application also includes a method for treating diseases, disorders or conditions that benefit from treatment with racemic 1,3-butanediol or R-1,3-butanediol, the method comprising administering an effective amount of one or more compositions of any one of claims 1 to 13 to a subject in need thereof.

The present application also includes a method for reducing alcohol consumption, for treating alcoholism, and/or for treating alcohol abuse comprising administering an effective amount of one or more compositions of any one of claims 1 to 13 to a subject in need thereof.

The present application also includes a method of initiating and/or maintaining ketosis comprising administering an effective amount of one or more compositions of any one of claims 1 to 13 to a subject in need thereof.

The present application also includes a method of reducing negative side effects of R-1,3-butanediol comprising administering an effective amount of one or more compositions of any one of claims 1 to 13 to a subject in need thereof.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### BRIEF DESCRIPTION OF THE DRAWINGS

The present application will be described in greater detail with reference to the attached drawings in which:

FIG. 1 is a graph showing the composite tolerability score for 6 participants, 60 minutes following ingestion of either a beverage comprising exemplary non-racemic 1,3-butanediol composition 5-a, or a beverage comprising comparative R-1,3-butanediol composition 2 (n=3 per group).

FIG. 2 is a graph showing composite BAES-STIM or BAES-SED scores for 6 participants 60 minutes following ingestion of either a beverage comprising exemplary non-racemic 1,3-butanediol composition 5-a, or a beverage comprising comparative R-1,3-butanediol composition 2 (n=3 per group).

### DETAILED DESCRIPTION

### 1. Definitions

Unless otherwise indicated, the definitions and embodiments described in this and other sections are intended to be applicable to all embodiments and aspects of the present application herein described for which they are suitable as would be understood by a person skilled in the art.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “composition of the application” as used herein refers to any composition comprising a non-racemic mixture of R-1,3-butanediol and S-1,3-butanediol as described herein.

The term “consisting” and its derivatives, as used herein, are intended to be closed terms that specify the presence of the stated features, elements, components, groups, integers, and/or steps, but exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end-result is not significantly changed. These terms of degree should be construed as including a deviation of at least ±5% of the modified term if this deviation would not negate the meaning of the word it modifies.

As used in this application, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The term “and/or” as used herein means that the listed items are present, or used, individually or in combination. In effect, this term means that “at least one of” or “one or more” of the listed items is used or present.

The term “subject” as used herein includes all members of the animal kingdom including mammals, and suitably refers to humans. Thus, the methods of the present application are applicable to both human therapy and veterinary applications.

The term “1,3-butanediol” as used herein refers to the racemic compound having the following chemical structure:

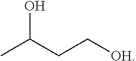

1,3-Butanediol is also known as 1,3-butylene glycol, butane-1,3-diol and 1,3-dihydroxybutane.

The term “R-1,3-butanediol” as used herein refers to a compound having the chemical formula:

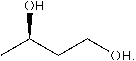

The term “S-1,3-butanediol” or as used herein refers to a compound having the chemical formula:

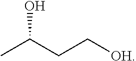

The term “pharmaceutical composition” as used herein refers to a composition of matter for pharmaceutical use.

The term “for pharmaceutical use” means compatible with the treatment of subjects.

The term “pharmaceutically acceptable” means compatible with the treatment of subjects.

The term “pharmaceutically acceptable salt” means an acid addition salt which is suitable for, or compatible with the treatment of subjects.

The term “nutraceutical” as used herein refers to any food or food ingredient (or additive) considered to provide medical or health benefits.

The term “solvate” as used herein means a compound, or a salt of a compound, wherein molecules of a suitable solvent are incorporated in the crystal lattice. A suitable solvent is physiologically tolerable at the dosage administered.

The term a “effective amount” of compositions of the application or as used herein refers to a quantity sufficient to, when administered to a subject, effect beneficial or desired results, including clinical results, and, as such, a “therapeutically effective amount” or synonym thereto depends upon the context in which it is being applied. Therefore, as used herein “therapeutically effective amount” is intended to mean that amount of a compound or composition that is sufficient to treat, prevent or inhibit diseases or conditions. The amount of a given compound or composition of the present application that will correspond to such an amount will vary depending upon various factors, such as the given compound or composition, the pharmaceutical formulation, the route of administration, the type of disease or disorder, the identity of the subject being treated, and the like, but can nevertheless be routinely determined by one skilled in the art.

The term “administered” as used herein means administration of a therapeutically effective amount of a composition of the application, a pharmaceutically acceptable salt and/or solvate thereof, to a cell either in cell culture or in a subject.

The terms “to treat”, “treating” and “treatment” as used herein and as is well understood in the art, means an approach for obtaining beneficial or desired results, including clinical results. Examples of beneficial or desired clinical results with respect to any disease, disorder or condition, include, but are not limited to diminishment of extent, stabilized (i.e., not worsening) state, preventing spread, delay or slowing of progression, amelioration or palliation of the state, and remission (whether partial or total), whether detectable or undetectable. “To treat”, “treating” and “treatment” can also mean prolonging survival as compared to expected survival if not receiving treatment. “To treat”, “treating” and “treatment” as used herein also includes prophylactic treatment.

“Palliating” a disease, disorder or condition means that the extent and/or undesirable clinical manifestations of a disease, disorder or condition are lessened and/or time course of the progression is slowed or lengthened, as compared to not treating the disease, disorder or condition.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The term “enantiomeric equivalents” as used herein refers to the molar amount of each enantiomer of butanediol. Therefore the percent of enantiomeric equivalents of each of R-1,3-butanediol and S-1,3-butanediol is defined by the molar quantity of either R-1,3-butanediol and S-1,3-butanediol divided by the total molar quantity of both R-1,3-butanediol and S-1,3-butanediol.

The term “composition of the application” as used herein refers to non-racemic mixtures of R-1,3-butanediol and S-1,3-butanediol wherein the R-1,3-butanediol is present in the composition in a greater amount by enantiomeric equivalents, relative to the S-1,3-butanediol as described herein.

### II. Compositions of the Application

1,3-Butane-diol has been shown to be safe for human consumption. R-1,3-Butanediol is a hypoglycemic agent and ingestion is associated with reduced glucose levels in the blood. Racemic 1,3-butanediol appears to cause hypoglycemia to a lesser extent, possibly due the presence of S-1,3-butanediol. Accordingly the present application is directed to compositions comprising R-1,3-butanediol comprising an amount of S-1,3-butanediol that lessens the negative effects of R-1,3-butanediol.

Therefore, the present application includes a composition comprising a non-racemic mixture of R-1,3-butanediol and S-1,3-butanediol wherein the R-1,3-butanediol is present in the composition in a greater amount by enantiomeric equivalents, relative to the S-1,3-butanediol.

In some embodiments, the composition comprises about 70% to about 99% by enantiomeric equivalents of the R-1,3-butanediol, and about 1% to about 30% by enantiomeric equivalents of the S-1,3-butanediol.

In some embodiments, the composition comprises about 70% to about 79.9% by enantiomeric equivalents of the R-1,3-butanediol, and about 20.1% to about 30% by enantiomeric equivalents of the S-1,3-butanediol.

In some embodiments, the composition comprises about 70% to about 75% by enantiomeric equivalents of the R-1,3-butanediol, and 25% to about 30% by enantiomeric equivalents of the S-1,3-butanediol.

In some embodiments, the composition comprises about 75% to about 79.9% by enantiomeric equivalents of the R-1,3-butanediol, and about 20.1% to about 25% by enantiomeric equivalents of the S-1,3-butanediol.

In some embodiments, the composition comprises about 75% to about 85% by enantiomeric equivalents of the R-1,3-butanediol, and about 15% to about 25% by enantiomeric equivalents of the S-1,3-butanediol.

In some embodiments, the composition comprises about 80% to about 89.9% by enantiomeric equivalents of the R-1,3-butanediol, and about 10.1% to about 20% by enantiomeric equivalents of the S-1,3-butanediol.

In some embodiments, the composition comprises about 80% to about 85% by enantiomeric equivalents of the R-1,3-butanediol, and about 15% to about 20% by enantiomeric equivalents of the S-1,3-butanediol.

In some embodiments, the composition comprises about 85% to about 89.9% by enantiomeric equivalents of the R-1,3-butanediol, and about 10.1% to about 15% by enantiomeric equivalents of the S-1,3-butanediol.

In some embodiments, the composition comprises about 83% to about 87% by enantiomeric equivalents of the R-1,3-butanediol, and about 17% to about 13% by enantiomeric equivalents of the S-1,3-butanediol. In some embodiments, the composition comprises about 85% by enantiomeric equivalents of the R-1,3-butanediol, and about 15% by enantiomeric equivalents of the S-1,3-butanediol.

In some embodiments, the composition comprises about 90% to about 99% by enantiomeric equivalents of the R-1,3-butanediol, and about 1% to about 10% by enantiomeric equivalents of the S-1,3-butanediol.

In some embodiments, the composition comprises about 90% to about 95% by enantiomeric equivalents of the R-1,3-butanediol, and about 5% to about 10% by enantiomeric equivalents the S-1,3-butanediol.

In some embodiments, the composition comprises about 95% to about 99% by enantiomeric equivalents of the R-1,3-butanediol, and about 1% to about 5% by enantiomeric equivalents of the S-1,3-butanediol.

In some embodiments, the composition comprises about 70%, about 75%, about 80%, about 85% or about 90% by enantiomeric equivalents of the R-1,3-butanediol, and about 30%, about 25%, about 20%, about 15% or about 10%, respectively by enantiomeric equivalents of the S-1,3-butanediol.

In some embodiments, the R-1,3-butanediol and S-1,3-butanediol, are present in the compositions in effective amounts. In some embodiments, the R-1,3-butanediol is present in an amount effective to act as an alcohol substitute, maintain ketosis, and/or to provide an alternative fuel source for the body and the S-1,3-butanediol is present in an amount effective to reduce the associated negative side effects of R-1,3-butanediol.

In some embodiments, the negative side effects of R-1,3-butanediol are selected from, but not limited to, one or more of hyperglycemia, stomach upset and gastrointestinal upset. In some embodiments, the negative side effects of R-1,3-butanediol are selected from, but not limited to, one or more of stomach upset and gastrointestinal upset.

In some embodiments, the stomach upset or gastrointestinal upset includes stomach cramps, stomach rumbling and/or nausea. In some embodiments, the stomach upset includes stomach cramps, stomach rumbling and/or nausea. In some embodiments, the negative side effects of R-1,3-butanediol are selected from gas or flatulence and stomach rumbling.

In some embodiments, the compositions of the application are used in, or are components of, consumable products, such as consumable beverages. Therefore, the present application also includes a consumable product comprising a composition of the application.

In some embodiments, the consumable product is a nutraceutical. Therefore, in some embodiments, the compositions of the application are nutraceutical compositions. In some embodiments, the nutraceutical composition is a nutraceutical beverage. In some embodiments, the nutraceutical beverage is formulated as a small serving or “shot”. In some embodiments, the nutraceutical composition is formulated as a gelatinous beverage such as a “Jello shot”.

In some embodiments, the consumable product is a beverage. In some embodiments, the beverage is any consumable liquid, including, but not limited to, water, carbonated water, naturally flavored water, artificially flavored water, soft drinks (sodas), teas, juices, ethanolic beverages and fermented beverages. In some embodiments, the beverage is an alcoholic beverage or a fermented beverage. In some embodiments, the beverage is beer, cider, wine, kombucha, spirits, hard soda, or the like.

In some embodiments, the compositions of the application are used as an alcohol substitute in the consumable product. Therefore, in some embodiments, the consumable beverage is an alcoholic drink. In some embodiments, the alcoholic drink is beer, wine or a spirit. In some embodiments, the alcoholic drink is formulated as a gelatinous beverage such as a “Jello shot”.

In some embodiments, the consumable beverage comprising a composition of the application is a ketogenic-friendly beverage for subjects on a ketogenic diet. In some embodiments, a ketogenic diet is a diet that comprises maintaining ketone bodies in the blood at levels, for example, above 0.5 mmol/L by restricting calories or carbohydrate intake. In some embodiments, the beverage comprising a composition of the application reduces glucose levels in the blood and counteracts the consumption of carbohydrates from the beverage. For example, drinking ethanol-based beverages can result in a significant drop in a person's ketone body level to, e.g., 0.2 mmol/L. In some embodiments, the person maintains or increase their ketone body level by drinking a beverage comprising a composition of the application.

In some embodiments, a consumable product comprising a composition of the application provides a temporary ketogenic effect that has benefits for weight loss and hunger suppression, as well as potential improvement in brain-function and increased physical performance.

Due to the appetite suppression nature of ketone bodies, a ketogenic friendly consumable product can provide people with a full feeling and prevent them from over-drinking or overeating. Based at least in part on this effect, in some embodiments, a beverage containing a composition of the application is safer than a corresponding beverage comprising ethanol. Unlike traditional alcoholic beverages that lower inhibitions and can lead to further unregulated drinking, a beverage comprising a composition of the application can provide a fuller and more content feeling at a lower quantity consumed than with ethanol and can help reduce the desire to drink more and become intoxicated. Therefore, in some embodiments, a consumable beverage comprising a composition of the application is useful for recovering alcoholics.

People who consume alcoholic drinks can on occasion suffer from so-called hangovers. In some embodiments, the consumable beverages comprising a composition of the application reduce the severity or even eliminate hangovers and side effects common from ethanol based alcoholic beverages.

In some embodiments, when the consumable beverage is a beer or wine, the beverage is made utilizing one or more of several steps. For example, in some embodiments, a beer is made by providing mash, boiling the wort, adding hops, and then fermenting the wort into a beer, as known in the brewing arts. In the case of wine, grapes are crushed and allowed to ferment. In some embodiments, ethanol from the fermentation is extracted and replaced with a composition of the application. In some embodiments, extraction is performed by boiling off the ethanol, which has a low boiling point of about 173° F. at sea level. In some embodiments, extraction is performed by fractional, or freeze, distillation, vacuum distillation, reverse-osmosis, or other methods for producing dealcoholized the beer. In some embodiments, a beer comprising a composition of the application is considered dealcoholized, or nonalcoholic, when the ethanol content is less than 0.5 percent alcohol by volume. In some embodiments, the substitution step includes providing the same, more, or less composition of the application than the amount of extracted ethanol. In some embodiments, to improve flavor, several steps are performed before or after alcohol substitution, such as dry-hopping, flavor-masking agents, and/or flavoring agents. In some embodiments, a beer comprising a composition of the application is carbonated by injecting CO₂. Carbonation from CO₂ injection, while common and often acceptable, can cause metallic or sour flavors in some beverages. Therefore, in some embodiments, to further improve the taste of the beer, carbonation is performed alternatively through a second fermentation. In some embodiments, the beer is carbonated with the addition of sugar and starter yeast. Very little ethanol is produced in the first 24-72 hours of this second fermentation but CO₂ can be produced. In some embodiments, the yeast is specifically chosen from cultures known to produce little alcohol or that are alcohol intolerant. In some embodiments, a beer is brewed as described above. However, before the fermentation process achieves 0.5 percent alcohol by volume (ABV), fermentation is halted and a composition of the application added to the beer to achieve a desired ABV percentage. In some embodiments, the consumable beverage is a spirit. In some embodiments substantially pure compositions of the application, or a high-content solution comprising a composition of the application, is utilized for mixing cocktails. In some embodiments, the high-content solution comprises a composition of the application and water. In some embodiments, the solution is aged with wood and/or include flavor additives. A benefit of such cocktails is that they can provide pleasant intoxicating effects while also providing health benefits of a ketogenic diet, for example by counteracting the carbohydrates present in most cocktail recipes and by maintaining ketosis. In some embodiments, such alcoholic beverages assist in weight loss, either directly or from appetite suppression. In some embodiments, the consumable beverage comprising a composition of the application is provided as a low carbohydrate or a zero-carbohydrate beverage. In one example, a low carbohydrate beverage includes beverages having a carbohydrate total of less than 10 g per serving. All natural low calorie sweeteners such as Xylitol, Erythritol, monk fruit, and/or stevia and artificial sweeteners such as sucrose, sucralose, acesulfame potassium, aspartame, saccharin can be utilized at a suitable concentration typical in beverage formulations. In some embodiments, the beverage is formulated as a low calorie beverage, such as a beverage having less than 100 calories per serving. In some embodiments, the consumable product further comprises preservatives, color, flavors and spices, flavor enhancers, emulsifiers, and/or stabilizers flavoring agents are selected from natural flavoring substances, nature-identical flavoring substances, artificial flavoring substances, diacetyl, acetylpropionyl, acetoin, isoamyl acetate, benzaldehyde, cinnamaldehyde, ethyl propionate, methyl anthranilate, limonene, ethyl decadienoate, allyl hexanoate, ethyl maltol, ethylvanillin, methyl salicylate, manzate, glutamic acid, glycine salts, guanylic acid, inosinic acid, acetic acid, malic acid, citric acid, ascorbic acid, fumaric acid, and lactic acid, and mixtures thereof. In some embodiments, the coloring agents are added to provide a favorable color to the consumable product. In some embodiments, the compositions of the application are present in the consumable product in an amount of about 0.1 wt % to about 40 wt %, about 1 wt % to about 40 wt %, about 10 wt % to about 40 wt %, about 20 wt % to about 40 wt %, about 25 wt % to about 40 wt %, 0.1 wt % to about 20 wt %, about 1 wt % to about 15 wt % or about 1 wt % to about 10 wt %.

The R- and S-1,3-butanediol are both available from numerous commercial sources (see for example the entries for each of these molecules in the PubChem™ database—https://pubchem.ncbi.nlm.nih.gov/). In some embodiments, R-1,3-butanediol and 1,3-butanediol (Brontide®) are available from Genomatica (San Diego, California, U.S.A.)

### III. Methods and Uses of the Application

The present application also includes a method for treating diseases, disorders or conditions that benefit from treatment with racemic 1,3-butanediol or R-1,3-butanediol, the method comprising administering an effective amount of one or more compositions of the application to a subject in need thereof. The present application further includes a use of one or more pharmaceutical compositions of the application for treating diseases, disorders or conditions that benefit from treatment with racemic 1,3-butanediol or R-1,3-butanediol, a use of one or more compositions of the application for the preparation of a medicament for treating diseases, disorders or conditions that benefit from treatment with racemic 1,3-butanediol or R-1,3-butanediol, as well as one or more compositions of the application for use to treat diseases, disorders or conditions that benefit from treatment with racemic 1,3-butanediol or R-1,3-butanediol.

The present application also includes a method for reducing alcohol consumption, for treating alcoholism, and/or for treating alcohol abuse comprising administering an effective amount of one or more compositions of the application to a subject in need thereof. The present application further includes a use of one or more pharmaceutical compositions of the application for reducing alcohol consumption, for treating alcoholism, or for treating alcohol abuse; a use of one or more compositions of the application for the preparation of a medicament for reducing alcohol consumption, for treating alcoholism, and/or for treating alcohol abuse as well as one or more compositions of the application for use to reduce alcohol consumption, to treat alcoholism, and/or to treat alcohol abuse.

In some embodiments, R-1,3-butanediol is present in the one or more compositions of the application in an amount effective to act as an alcohol substitute.

The present application also includes a method of initiating and/or maintaining ketosis comprising administering an effective amount of one or more compositions of the application to a subject in need thereof. The present application further includes a use of one or more compositions of the application for initiating and/or maintaining ketosis, a use of one or more compositions of the application for the preparation of a medicament for initiating and/or maintaining ketosis as well as one or more compositions of the application for use to initiate and/or maintain ketosis.

In some embodiments, the initiating and/or maintaining ketosis suppresses appetite and/or promotes weight loss. Accordingly, in some embodiments, the one or more compositions of the application can be used or administered for suppressing appetite and/or promoting weight loss.

The present application also includes a method of reducing negative side effects of R-1,3-butanediol comprising administering an effective amount of one or more compositions of the application to a subject in need thereof. The present application further includes a use of one or more compositions of the application for reducing negative side effects of R-1,3-butanediol, a use of one or more compositions of the application for the preparation of a medicament for reducing negative side effects of R-1,3-butanediol as well as one or more compositions of the application for use to reduce adverse side effects of R-1,3-butanediol.

In some embodiments, the negative side effects of R-1,3-butanediol are selected from, but not limited to, one or more of hyperglycemia, stomach upset and gastrointestinal upset. In some embodiments, the negative side effects of R-1,3-butanediol are selected from, but not limited to, one or more of stomach upset and gastrointestinal upset.

In some embodiments, the stomach upset or gastrointestinal upset includes stomach cramps, stomach rumbling and/or nausea. In some embodiments, the stomach upset includes stomach cramps, stomach rumbling and/or nausea. In some embodiments, the negative side effects of R-1,3-butanediol are selected from gas or flatulence and stomach rumbling.

### EXAMPLES

The following non-limiting examples are illustrative of the present application:

### Example 1: Exemplary Compositions of the Application

Exemplary Composition 1: 90-99% R-1,3-butanediol, 1-10% S-1,3-butanediol
Exemplary Composition 2: 80-89.9% R-1,3-butanediol, 10.1%-20% S-1,3-butanediol
Exemplary Composition 3: 70-79.9% R-1,3-butanediol, 20.1-30% S-1,3-butanediol.
Exemplary Composition 4: 90-99% R-1,3-butanediol, 1-10% S-1,3-butanediol.
Exemplary Composition 5: 80-89.9% R-1,3-butanediol, 10.1%-20% S-1,3-butanediol.
Exemplary Composition 5-a: 85% R-1,3-butanediol, 15% S-1,3-butanediol.
Exemplary Composition 6: 70-79.9% R-1,3-butanediol, 20.1-30% S-1,3-butanediol.
Exemplary Composition 6-a: 75% R-1,3-butanediol, 25% S-1,3-1,3-butanediol.
Comparative Composition 1: Racemic 1,3-butanediol
Comparative Composition 2: R-1,3-butanediol (>99% R enantiomer)
Comparative Composition 3: S-1,3-butanediol (>99% S enantiomer)

Percentages are provided by enantiomeric equivalents. For example, exemplary Composition 5-a comprises 85% by enantiomeric equivalents of R-1,3-butanediol and 15% by enantiomeric equivalents of S-1,3-butanediol.

All exemplary compositions are prepared by combining the R- and S-enantiomers in the required ratios and mixing with a suitable liquid, such as water, carbonated water, naturally flavored water, artificially flavored water, soft drinks (sodas), teas, juices, ethanol-free beer, ethanol-free wine or fermented beverages. Flavourings are further optionally added.

### Example 2: Testing

Exemplary compositions of the application have been administered to human subjects and their subjective experience, their assessment of the taste and enjoyability and their tolerability (e.g. stomach and gastrointestinal upset) has been recorded and evaluated compared to the comparative compositions.

### Example 2.1: An Acute Pilot Consumer Study to Assess the Subjective Effects of an Exemplary Composition Comprising a Non-Racemic Ratio of R- and S-1,3-Butanediol Compared to a Comparative Composition Comprising R-1,3-Butanediol in an Adult Population

### Introduction

1,3-Butanediol (1,3-butylene glycol) is known to be a chemical of low toxicological concern, with uses as a flavouring agent and solvent and humectant for food additives, cosmetics and other products. Recently there has been interest in its use as a performance enhancer, and for its euphoric effects.

Studies examining the potential for 1,3-butanediol to act as a meal replacement indicated that 1,3-butanediol was of low toxicological concern. European Chemicals Agency (ECHA) registration dossiers indicate acute oral, inhalation and dermal toxicity was low, with no adverse events observed in the registered studies. Scala and Paynter fed rats 1,3-butanediol at dietary levels of 1.0, 3.0, and 10% for two years, and dogs were fed the compound at dietary levels of 0.5, 1.0, and 3.0% for the same period, there were no adverse effects attributable to the ingestion of 1,3-butanediol in either species (Scala, R. A., and O. E. Paynter. 1967. “Chronic Oral Toxicity of 1,3-Butanediol.” Toxicology and Applied Pharmacology 10 (1): 160-64).

Several studies have investigated the impacts of acute single or sub-chronic doses with human volunteers. Tobin et al reported three studies of 1,3-butanediol ingestion, indicating the compound was well tolerated, and showed no significant changes in physiologic endpoints (Tobin, R. B., et al., 1975. “Nutritional and Metabolic Studies in Humans with 1,3-Butanediol.” Federation Proceedings 34 (12): 2171-76). More recent studies have focused on subjective effects of ingestion, as 1,b-Butanediol is known to cause euphoria and gastrointestinal issues (Frye, G. D., et al., 1981. The Journal of Pharmacology and Experimental Therapeutics 216 (2): 306. Majchrowicz, E., et al., 1976. Science 194 (4270): 1181-82). To date, clinical studies have shown bolus acute and sub-chronic doses increase blood beta-hydroxybutyrate (BHB) concentrations with minimal gastrointestinal (GI) issues (Scott, B, E., et al. 2019. Journal of Science and Medicine in Sport/Sports Medicine Australia 22 (6): 702-6.). A study on cycling performance showed bolus doses of 0.5 g/kg body weight (bw) or 0.7 g/kg bw lead to nausea, euphoria and dizziness, with less impact splitting the amount into 2 doses of 0.35 g/kg bw administered 1.5 hr apart (Shaw D. M., et al. 2019. International Journal of Sport Nutrition and Exercise Metabolism 29 (5): 466-73. For a 70 kg individual, 0.7 g/kg bw equates to consumption of 49 g of 1,3-butanediol in a day. No information regarding the enantiomeric purity of the 1,3-butanediol was reported for these studies.

A study on a novel ketone ester, bis-hexanoyl (R)-1,3-butanediol (BH-BD), which is hydrolyzed into hexanoic acid and R-1,3-butanediol, showed only mild side effects and no adverse events of ingesting 25 g/day for 28 consecutive days (Chen, O. T., et al. 2021. Nutrients 13 (6): 2066.; Stubbs B. J. et al. 2021. Food and Chemical Toxicology: An International Journal Published for the British Industrial Biological Research Association 147 (January): 111859.). Headache, nausea, and dizziness were most commonly described among the treatment group, with no clinically meaningful changes in physiologic results from baseline. Importantly, the only significant difference in Brief-Biphasic Alcohol Effects stimulation or sedation scores was observed only on the first day, when 12.5 g of BH-BD was administered.

The present pilot study examined the subjective impact of ingesting either a beverage comprising exemplary non-racemic 1,3-butanediol composition 5-a, or a beverage comprising comparative R-1,3-butanediol composition 2 using two subjective and validated questionnaires examining alcohol-like effects as well as tolerability.

### Methods

This consumer pilot trial was an acute study of the effects following consumption of 20 g of either a beverage comprising exemplary non-racemic 1,3-butanediol composition 5-a (85% by enantiomeric equivalents R-1,3-butanediol, 15% by enantiomeric equivalents S-1,3-butanediol), or a beverage comprising comparative R-1,3-butanediol composition 2. Two scales were utilized: subjective effects with Biphasic Alcohol Effects Scale (BAES), a validated tool to measure the effects of drinking alcohol (Martin, C. S., et al. 1993. Alcoholism, Clinical and Experimental Research 17 (1): 140-46), and GI Tolerance with a Modified Beverage Tolerability Questionnaire (m-BTQ; Chen., 0. T., et al. 2021. Nutrients 13 (6): 2066). Participants were asked to fast for at least 2 hours before consuming the product, and complete the m-BTQ and BAES at 60 minutes following the consumption of the product.

The study population was 6 individuals between 25-40 years old. Questionnaires for the m-BTQ and BAES are provided below in Tables 1 and 2.

TABLE 1
Modified Beverage Tolerability Questionnaire (m-BTQ)
In the past hour, have you experienced any of these symptoms:

| Check the box of the | None or no | | | |
|---|---|---|---|---|
| severity of your | change | Mild | Moderate | Severe |
| symptoms | [0] | [1] | [2] | [3] |

Gas or Flatulence
Nausea
Vomiting
Burping
Reflux
Headache
Dizziness
Abdominal cramping
Stomach rumbling
Diarrhea

TABLE 2
Biphasic Alcohol Effects Score (BAES) Questionnaire
The following adjectives describe feelings that some people have
after drinking alcohol. Please rate the extent to which drinking
alcohol has produced these feelings in you at the present time.
1) Difficulty Concentrating

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

2) Down

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

3) Elated

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

4) Energized

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

5) Excited

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

6) Heavy Head

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

7) Inactive

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

8) Sedated

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

9) Slow thoughts

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

10) Sluggish

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

11) Stimulated

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

12) Talkative

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

13) Up

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

14) Vigorous

| | | | | | | | | | | |
|---|---|---|---|---|---|---|---|---|---|---|
| 0 | 1 | 2 | 3 | 4 | 5 | 6 | 7 | 8 | 9 | 10 |
| (not at all) | | | | | | | | | | (extremely) |

### Results

Participants were randomized into exemplary non-racemic 1,3-butanediol composition 5-a or comparative R-1,3-butanediol composition 2 groups and all completed the study protocol. The six participants were between 25-40 years old, were 100% Caucasian and all (100%) identified as male.

Table 3 summarizes the results from the two questionnaires, performed 60 minutes following consumption. Composite tolerability scores for the exemplary non-racemic 1,3-butanediol composition 5-a beverage were lower than that of the comparative R-1,3-butanediol composition 2 beverage, indicating the exemplary non-racemic composition may be more tolerable than comparative R-1,3-butanediol composition (Table 3). “Gas or Flatulence” and “Stomach rumbling” were the two categories which appeared to have the strongest effects of both beverages for the participants. Composite BAES scores indicated the two beverages had similar stimulatory and sedentary subjective effects (Table 3).

TABLE 3
Mean Composite Tolerability Score (m-BTQ) and Mean
STIM/SED Scores (BAES) for the participants 60
minutes following consumption of the beverage.

| | Mean | Mean | Mean |
|---|---|---|---|
| | Composite | BAES-STIM | BAES-SED |
| | Tolerability | Composite | Composite |
| | Score | Score | Score |
| Exemplary non-racemic 1,3- | 4.3 | 43 | 21.3 |
| butanediol composition 5-a | | | |
| Comparative R-1,3- | 8 | 41.7 | 22.3 |
| butanediol composition 2 | | | |

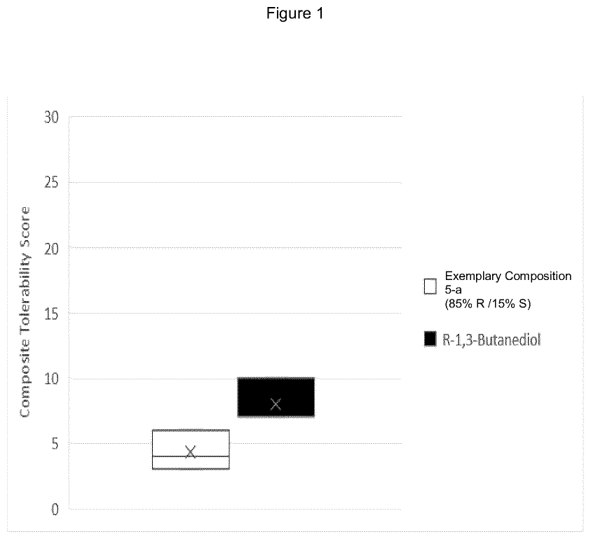

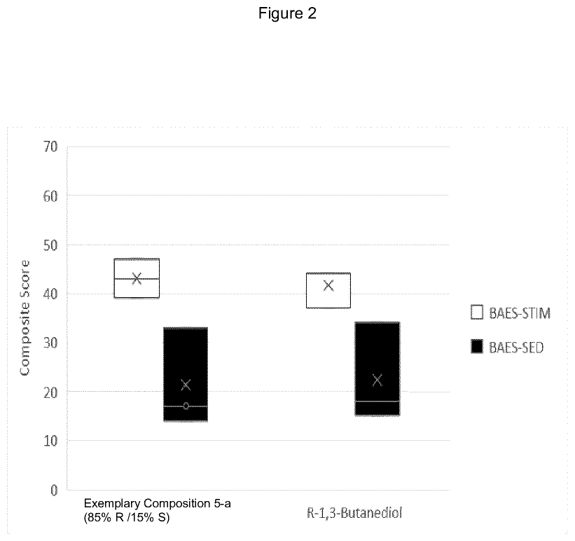

Due to the initial small sample size of this pilot study, statistical analysis of these results were not conducted. FIGS. 1 and 2 are graphs showing representative figures for the population.

### Discussion

In this preliminary study comparing acute use of exemplary non-racemic 1,3-butanediol composition 5-a and comparative R-1,3-butanediol composition 2 on tolerability, sedation and stimulation, it was found that both compositions appear to have similar effects. Both products were well tolerated by all participants and caused relatively minor side effects. However, it appears that exemplary non-racemic 1,3-butanediol composition 5-a produces less gastrointestinal issues, as was indicated by it having a lower BTQ composite score. GI issues are known side effects of consuming 1,3-butanediol preparations (McCarthy, C. G., et al. 2021. The Journal of Pharmacology and Experimental Therapeutics 379 (3): 245).

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A composition comprising a non-racemic mixture of R-1,3-butanediol and S-1,3-butanediol wherein the R-1,3-butanediol is present in the composition in a greater amount by enantiomeric equivalents, relative to the S-1,3-butanediol.

2. (canceled)

3. The composition of claim 1, wherein the composition comprises about 70% to about 79.9% by enantiomeric equivalents of the R-1,3-butanediol, and about 20.1% to about 30% by enantiomeric equivalents of the S-1,3-butanediol.

4. The composition of claim 1, wherein the composition comprises about 70% to about 75% by enantiomeric equivalents of the R-1,3-butanediol, and 25% to about 30% by enantiomeric equivalents of the S-1,3-butanediol.

5. The composition of claim 1, wherein the composition comprises about 75% to about 79.9% by enantiomeric equivalents of the R-1,3-butanediol, and about 20.1% to about 25% by enantiomeric equivalents of the S-1,3-butanediol.

6. The composition of claim 1, wherein the composition comprises about 80% to about 89.9% by enantiomeric equivalents of the R-1,3-butanediol, and about 10.1% to about 20% by enantiomeric equivalents of the S-1,3-butanediol.

7. The composition of claim 1, wherein the composition comprises about 80% to about 85% by enantiomeric equivalents of the R-1,3-butanediol, and about 15% to about 20% by enantiomeric equivalents of the S-1,3-butanediol.

8. The composition of claim 1, wherein the composition comprises about 85% to about 89.9% by enantiomeric equivalents of the R-1,3-butanediol, and about 10.1% to about 15% by enantiomeric equivalents of the S-1,3-butanediol.

9. (canceled)

10. The composition of claim 1, wherein the composition comprises about 90% to about 95% by enantiomeric equivalents of the R-1,3-butanediol, and about 5% to about 10% by enantiomeric equivalents the S-1,3-butanediol.

11. (canceled)

12. (canceled)

13. The composition of claim 1, wherein the R-1,3-butanediol and S-1,3-butanediol are present in the composition in effective amounts to act as an alcohol substitute, maintain ketosis, and/or to provide an alternative fuel source for the body.

14. The composition of claim 1, wherein the S-1,3-butanediol is present in an amount effective to reduce the associated negative side effects of R-1,3-butanediol.

15. The composition of claim 14, wherein the associated negative side effects of R-1,3-butanediol are selected from one or more of stomach upset and gastrointestinal upset.

16. A consumable product comprising a composition of claim 1.

17. The consumable product of claim 16, wherein the consumable product is a beverage.

18. The consumable product of claim 17, wherein the beverage is water, carbonated water, naturally flavored water, artificially flavored water, soft drinks (sodas), teas, juices, ethanolic beverages and fermented beverages.

19. (canceled)

20. (canceled)

21. (canceled)

22. The consumable product of claim 16, wherein the composition is a substitute for alcohol in the consumable product, or the composition is a nutraceutical.

23. (canceled)

24. The consumable product of claim 16, wherein the compositions is present in the consumable product in an amount of about 0.1 wt % to about 40 wt %, about 1 wt % to about 40 wt %, about 10 wt % to about 40 wt %, about 20 wt % to about 40 wt %, about 25 wt % to about 40 wt %, 0.1 wt % to about 20 wt %, about 1 wt % to about 15 wt % or about 1 wt % to about 10 wt %.

25. A method for treating diseases, disorders or conditions that benefit from treatment with racemic 1,3-butanediol or R-1,3-butanediol, the method comprising administering an effective amount of one or more compositions of claim 1 to a subject in need thereof.

26. A method for reducing alcohol consumption, for treating alcoholism, and/or for treating alcohol abuse comprising administering an effective amount of one or more compositions of claim 1 to a subject in need thereof.

27. A method of initiating and/or maintaining ketosis comprising administering an effective amount of one or more compositions of claim 1 to a subject in need thereof.

28. A method of reducing negative side effects of R-1,3-butanediol comprising administering an effective amount of one or more compositions of any claim 1 to a subject in need thereof.
